(19) United States
(12) Patent Application Publication
(10) Pub. No.: US 2010/0170812 A1
(43) Pub. Date: Jul. 8, 2010

(54) LOW PROFILE SUTURE NEEDLE HOLDER

(76) Inventor: David Odierno, Weymouth, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 12/601,183

(22) PCT Filed: May 30, 2008
(86) PCT No.: PCT/US08/06835

§ 371 Date: Nov. 20, 2009

Related U.S. Application Data
(60) Provisional application No. 60/932,432, filed on May 31, 2007.

(51) Int. Cl. A61B 17/06

(52) U.S. Cl. 206/63.3

(57) ABSTRACT

Devices and methods for removing and storing suture needles from a suturing device utilize a body containing one or more slots for holding suture needles. Additional embodiments can include a removable protective cover and a base for standing the needle holding device on a surface. The devices and methods are particularly suitable for improving the safety and convenience of removal and disposal of used suture needles from an endoscopic suturing device.

### CROSS REFERENCE TO RELATED APPLICATIONS

This application claims the benefit of U.S. Provisional Application No. 60/932,432, filed May 31, 2007 and entitled SUTURE NEEDLE HOLDER. The entire teachings of the above application is incorporated herein by reference.

### BACKGROUND OF THE INVENTION

The disposal of used surgical suture needles poses a number of problems. The needles are sharp and therefore pose a risk of puncture wounds to operating room personnel. Used suture needles also may be contaminated with biohazard material, such as blood which may carry blood-born pathogens including HCV and HIV. While various specialized biohazard disposal containers are available, the complexity and specialized operation of modern suturing devices, particularly those used in conjunction with endoscopy, can make it difficult or challenging to retrieve and secure used surgical needles such as suture needles.

There remains a need to develop specialized devices and methods for the safe and convenient retrieval, storage, and disposal of surgical needles.

### SUMMARY OF THE INVENTION

The present invention provides devices and methods for the removal and storage of surgical suture needles, particularly after their use in endoscopic surgery. The devices and methods according to the invention are safer and more convenient to use than those previously available.

The present invention is a low profile storage device for suture needles. In a preferred embodiment, the device includes a body or housing whose thickness allows it to fit between the arms of a suturing device containing a suture needle. The body portion operative to fit between the arms of a suturing device has a uniform thickness along a certain arc, cross-section or volume so as to facilitate movement by the user between the arms. The body of the storage device contains one or more slots that open at peripheral edge of the body. Each of the slots has a size that allows it to hold a suture needle. In certain embodiments of the suture needle storage device, at least one slot in the body includes two or more joined segments, and each pair of joined segments is connected at an angle. In preferred embodiments, the slot can include two segments, or three segments, or the slot can be T-shaped. In some embodiments at least one slot contains a central channel connecting with a plurality of side channels, and each of the side channels has a size suitable to hold a suture needle. In other embodiments, at least one slot contains one or more spaces disposed along its length, and each of the spaces has a size suitable to hold a suture needle. In another embodiment the body is wheel shaped and has a plurality of slots radially disposed around a central axis. The slots can be spaced along a circumferential length of the body such that the slots are equally spaced from each other. The storage device can hold and retain needles placed within the storage slots by means of compression by the needle of the surrounding body material, or by placement of the needle onto a patch of adhesive coating at a needle storage position within a storage slot.

In preferred embodiments, the body of the storage device can be fabricated with a foam, plastic, or rubber material. In some embodiments, the material is elastic. The body can also be fabricated from a composite of two or more materials. A preferred embodiment of the storage device has a body with a foam core. Some embodiments of the storage device include a movable cover which can be transparent, e.g., to permit the counting of needles used in a surgical procedure. The cover or closer device also can include an opening for the introduction of suture needles, and can optionally rotate around the body or be attached to the body via a hinge.

In a preferred suture needle storage device according to the invention, the suture needles placed into the device have been used in surgery performed with the aid of a suturing device, such as an endoscopic suturing device. Another preferred embodiment of the invention can comprise a kit that includes one or more storage devices and a suturing device.

Another aspect of the invention is a method for removing a suture needle from a suturing device. The method includes positioning a storage device according to the invention adjacent to the suturing device, sliding the suture needle into a slot on the storage device, and releasing the suture needle from the suturing device. The method can also include suturing a region of an organ or tissue during surgery with a suturing needle mounted on a suturing device. The method can further include inserting the storage device between suture needle holder elements of the suturing device. Thus, the needles can be removed from the suture device without the user having to manually touch or hold the needle, thereby substantially reducing the risk of a shin puncture during transfer. In some embodiments the method further includes discarding the storage device, for example in a biohazards waste receptacle, after one or more suture needles have been stored in the device. The method can also include opening a cover on the storage device, closing a cover on the storage device, or rotating or adjusting the cover so as to expose additional slots, channels, or spaces for the storage of subsequent needles. In certain embodiments of the method, the storage device contains at least one slot which has a two or more side channels or spaces for receiving suture needles, and two or more suture needles are stored in the device. In other embodiments, the storage device contains two, three or more slots, and two or more needles are stored in the device.

### BRIEF DESCRIPTION OF THE DRAWINGS

The invention will be more fully understood by referring to the Detailed Description of the Invention in conjunction with the Drawings, of which:

FIG. 1A provides an illustrative embodiment of a suture needle holder in accordance with the invention;

FIG. 1B provides an enlargement of one embodiment of a slot for receiving and storing suture needles in a suture needle holder according to the invention;

FIG. 1C provides an enlargement of another embodiment of a slot for receiving and storing suture needles in a suture needle holder according to the invention;

FIG. 1D provides an enlargement of another embodiment of a slot for receiving and storing suture needles in a suture needle holder according to the invention;

FIG. 1E shows an alternative to the embodiment shown in FIG. 1B.

FIG. 1F shows an alternative to the embodiment shown in FIG. 1C.

FIG. 1G shows an alternative to the embodiment shown in FIG. 1D.

FIG. 2A shows an illustrative embodiment of a suture needle holder in accordance with the invention;

FIG. 2B shows an enlargement of one embodiment of a compound slot for receiving and storing suture needles in a suture needle holder according to the invention;

FIG. 2C shows an enlargement of another embodiment of a compound slot for receiving and storing suture needles in a suture needle holder according to the invention;

FIG. 2D shows an enlargement of another embodiment of a compound slot for receiving and storing suture needles in a suture needle holder according to the invention;

FIG. 3 provides a schematic illustration of a wheel-shaped embodiment of a suture needle holder according to the invention;

FIG. 4A shows a schematic illustration of a suturing device with suture needle for use with a suture needle holder according to the invention;

FIG. 4B shows a schematic illustration of a suture needle holder according to the invention in juxtaposition with a suturing device for the removal of a suture needle into the device and storage.

### DETAILED DESCRIPTION

The present invention relates to novel devices for retrieval, storage, and disposal of surgical suture needles, and methods for using the devices, particularly in connection with endoscopic surgery. The devices and methods provided by the invention solve many of the problems associated with the use of surgical suturing devices, including the danger of removing used suture needles from such devices, transferring them to a storage container, and storing them until appropriate disposal in a hazardous waste container. The devices are particularly adapted to permit needle removal and protected storage in one operation without the need for the user touching the needle with their hands. In some embodiments, the devices also permit vizualization of stored needles for counting, so that the suture needles used in a given surgical procedure can be accounted for, or archived as a unit to provide a record of the procedure. Preferred embodiments of the suture needle storage device have storage for multiple suture needles, allowing all the needles used in a given surgical procedure to be conveniently stored in a single device. The devices are suitable to accept suture needles having a thickness in a range of about 0.1 mm to about 2 mm and lengths in a range of 1 mm to 30 mm or more. The devices can be designed to accept either a range of needle sizes and/or shapes within a single device or alternatively designed to accept a single needle design in a given device.

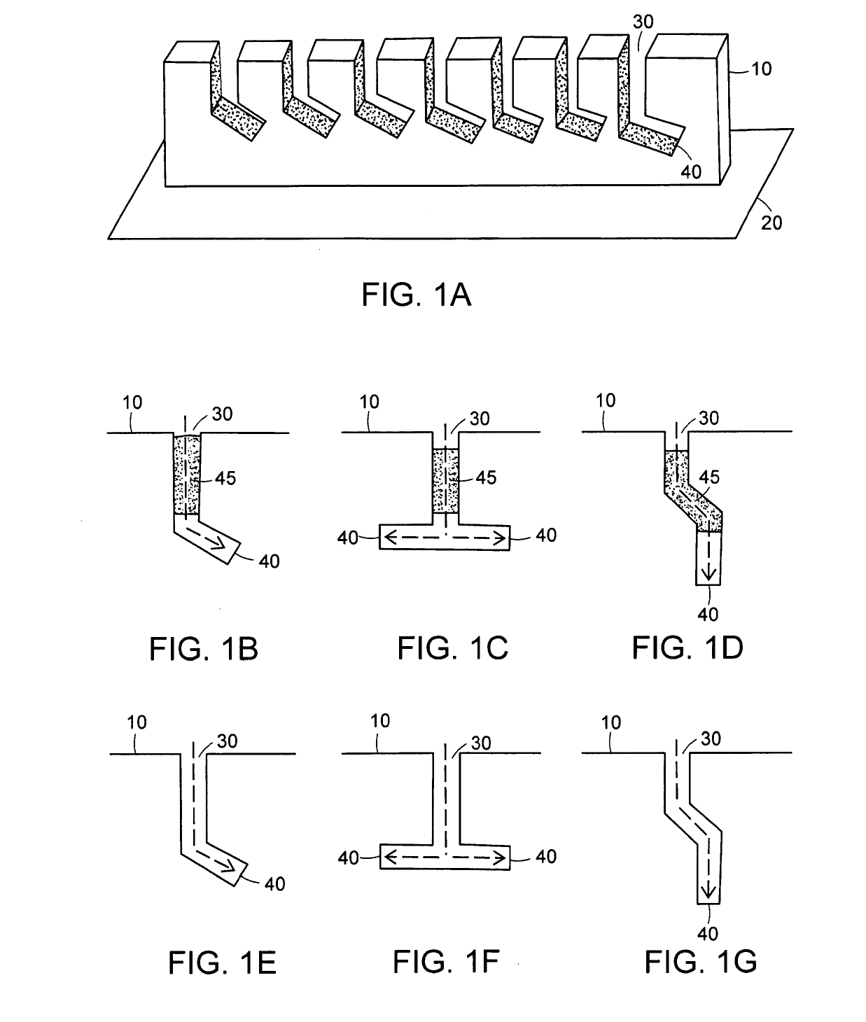

Referring to the illustrative embodiment of a suture needle storage device according to the invention shown in FIG. 1, the device includes a body 10 which can be attached to and supported by a base 20, resulting in, for example, a T-shaped profile suitable for standing the device on a flat surface during use. The body 10 has a thickness in a range of about 1 mm to about 30 mm or more and preferably has a thickness so that it can fit between the arms of a suturing device. The body 10 contains one or more slots 30 for receiving suture needles directly from a suturing device. Each slot 30 opens to an edge of the body 10, allowing a suture needle to be stored by sliding the needle, preferably while still attached to a suturing device, across the edge of the body and into the interior of the body for storage within the slot. In the embodiment illustrated in FIG. 1, the needle is passed through the slot to a needle receptacle 40 connected to the slot. The needle storage position 40 can be a specialized portion of the slot. For example, the needle receptacle or storage position 40 can be located at a terminal portion of the slot 30, for ease of storing a needle and for stabilizing the position of a needle within the storage device once removed from the suturing device.

A slot 30 can include two or more joined segments, and each pair of adjacent segments can be connected at an angle. The angular connections between slot segments can be abrupt or gradual, and may be formed by the intersection of two adjacent straight segments, or can be formed by a curved portion of the slot to form a bend. By including one or more angular connections or bends along the length of the slot, for example as illustrated in FIGS. 1B through 1G, the possible exit of the needle from the storage device after parking the needle is inhibited. For example, a needle slot 30 can be straight, L-shaped, T-shaped, J-shaped, S-shaped, Y-shaped, or zig-zagged. Slot shapes that have one or more angles or bends are preferred. Inclusion of multiple slot segments also provides needle receptacles 40 for multiple needles. Generally, each needle receptacle 40 can be located at the end of a terminal segment. A given slot 30 can be outfitted with 1-10 or more terminal segments and an equivalent number of needle storage positions 40. A suture needle storage device can possess 1-10 or more individual slots 30, so as to allow for a desired number of needles to be retrieved, stored, archived, or disposed of as a single unit.

In preferred embodiments, slot 30 and/or needle receptacle 40 are filled with a foam or other pliable material 45 having a slot cut down the middle (see, e.g., FIGS. 1B, 1C, and 1D). The needle can be passed along the slot and come to rest either within the foam 45, in which case the foam serves to frictionally engage the needle and hold it in place, or past the foam within needle receptacle 40, in which case the foam closes to trap the needle in the needle receptacle. Alternatively, the material of body 10 can serve to grasp or trap the stored needle directly (see, e.g., FIGS. 1E, 1F, and 1G). For example, the body can be fabricated of a compressible or foam material itself, and slots 30 can be of similar dimension to the needle thickness, or slightly less, so as to grasp the needle but still permit its placement within the slot.

A needle storage unit having a base for stable placement, e.g., on a table in a surgical area, conveniently uses slots open at the top edge of body 10. However, slots opening to a side of body 10 are also contemplated. A cover, such as a transparent removable cover, can optionally be added to a device such as depicted in FIG. 1A, allowing the stored needles to be stabilized and protected inside a closed container for storage or disposal without concern for needles falling out of the device.

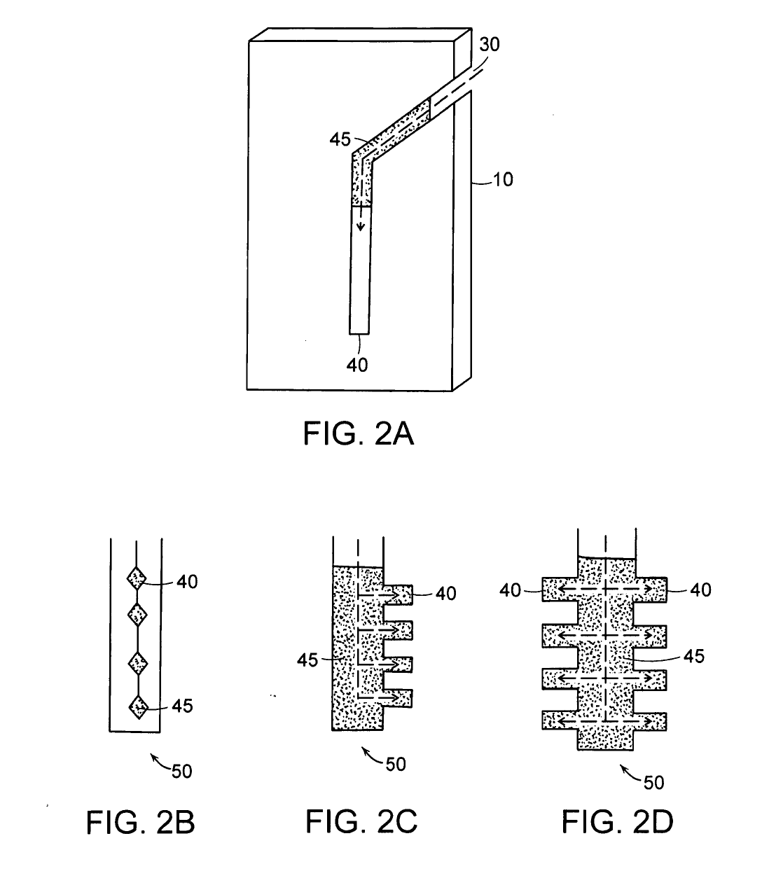

FIG. 2A illustrates an embodiment of the suture needle storage device that has a body 10 but no base and can be used, for example, as a hand-held device. This embodiment is illustrated with a single slot 30 with an opening to the side; however, two or more slots can also be employed with this device. The embodiment shown in FIG. 2A has a single needle receptacle or storage position 40, at the end of a slot segment joined at an angle to the open segment of slot 30. The device of FIG. 2A can be used, for example, in a single-use fashion, for the retrieval and storage of a single suture needle. A single-use device can be used without concern for the presence of other used needles within the device, which could under certain circumstances provide distraction or risk of used needles clogging the device or dropping out. Additional embodiments are shown in FIGS. 2B, 2C, and 2D, in which two or more needle receptacles 40 are added along the length of slot 30, resulting in the formation of a compound needle receptacle 50. A compound needle receptacle has a central channel that opens directly into 1-10 or more needle storage positions 40, or alternatively contains 1-10 or more spaces within the channel that serve as needle positions 40.

In the embodiment shown in FIG. 2B, the central channel is a portion of slot 30 that is narrower than the open segment of the slot. For example, the central channel can be narrowed to less than the diameter of the suture needles collected in the device, so as to restrict the movement of collected needles out of the channel once they are placed. One or more needle receptacles, each large enough to accommodate a single needle, are positioned within the central channel. Needle receptacle spaces 40 can be positioned in a serial or linear arrangement, as shown in FIG. 2B, or in another arrangement such as a branched or fenestrated alignment. The spaces themselves can have any shape and dimension that accepts the intended suture needle, such as, for example, a diamond, circle, square, or triangle shape or another shape. The size of a needle receptacle space is preferably close to the diameter of the needle, or slightly less, e.g., 1%, 2%, 3%, 5%, 10%, 20%, or 50% less than the diameter of the needle. Setting the size of the receptacle space slightly less than the diameter of the needle permits the needle to compress the body material once in place, giving rise to pressure against the needle to hold it in place; this is particularly true if the body material is somewhat elastic.

The embodiments shown in FIGS. 2C and 2D offer a number of needle storage positions 40 which are easily accessible from the central channel. Rectangular positions 40 are depicted, but they can have any shape that permits a needle to be held at the storage position. The size of the storage positions is similar to the needle diameter. As for other embodiments, if the size of the storage position is slightly less than the diameter of the needle, then the needle compresses the body material, giving rise to pressure against the needle. Alternatively, an adhesive patch can be added at the storage position to stabilize the needle once positioned in the receptacle. In the embodiments depicted in FIGS. 2C and 2D, the central channel can be open, for example having a size larger than the needle diameter, e.g., 1.5, 2, 2.5, 3, 4, 5 or more times the needle diameter. A preferred embodiment can have an opening size in a range of 0.1 mm to 3 mm. The openings or slots can have an insert material such as a foam 45 with a slit in which the needle can be manually inserted and retained by friction with the needle surface. A larger central channel provides easier access to individual needle receptacles. Stability of needle placement can be afforded by restricting the size of the needle receptacle, use of an adhesive at the receptacle, or by restricting the size of the slot near the opening to less than the size of the central channel.

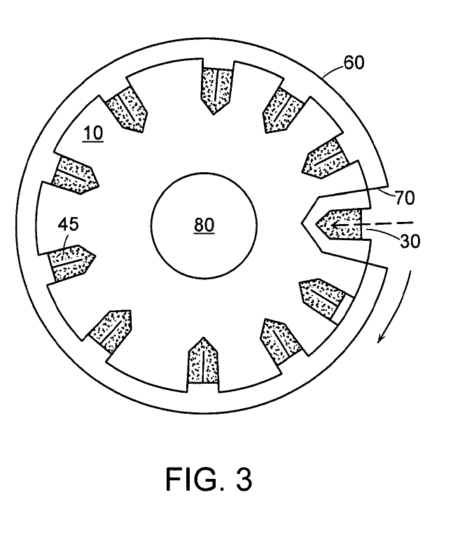

A wheel-shaped suture needle storage device is depicted in FIG. 3. In this embodiment, a body 10 has an overall circular or disc-shaped profile and one or more radially aligned slots 30 opening to the edge. An advantage of this arrangement is that the body can be outfitted with a cover 60, that can be rotated around the body to provide access to individual slots 30. In an alternative embodiment, one or more needle receptacles 40 can be placed directly at the edge of the wheel-shaped body 10, without the need for a slot 30, because rotation of the cover with respect to the body can prevent egress of already deposited needles from the device. One mechanism for providing relative movement between the body and the cover is to provide a rotation knob 80 which is attached to the body through a hole at the center of the cover. The cover can be held in one hand, or placed into a stand, and the other hand can actuate or rotate the knob and thus turn the wheel-shaped body within the cover, so as to secure a deposited needle and/or to expose a slot or needle storage position for placement of another needle. The cover can optionally be attached to the body through a mechanism that stabilizes each receptacle or slot when aligned with an opening or window 70 in the cover, for example by employing a clicking or ratcheting mechanism. Either one or both directions of rotation can be allowed by the mechanism, as desired. The closure device or cover can be transparent to allow visualization of the number of needles stored in the device, as well as of the number of remaining empty receptacles. The slot or receptacle can have any shape and dimension suitable for depositing and retaining the needle, such as a rectangular, triangular, conical, cylindrical, V-shaped, or other form. Preferably, the needle is held in position through the use of compression (i.e., the storage position size is about equal to or slightly smaller than the needle diameter) or an adhesive or an elastic material such as a foam 45 through which a needle can be inserted or a material with a slit so that the needle can slide therethrough.

The body of a suture needle storage device can be of any material that stably accepts a slot and needle receptacle as described above. Preferred materials include plastic (e.g., polystyrene, polyethylene, polycarbonate, acrylic or other plastic materials), foam, and rubber (either synthetic or natural). Preferred body materials are sufficiently elastic and compressible so as to allow a needle to be introduced into a space slightly smaller than the needle diameter, resulting in compression of the body material by the needle, and exertion of a force by the body material against the needle to hold the needle in place. Body material can be manipulated by any known method to provide one or more slots and needle receptacles, including cutting, milling, welding, gluing, fastening, and injection molding. Dyes or paints can be applied within material or on its surface to provide any desired color, markings, labeling, or writing on a body or a cover of the device to assist in using the device, for aesthetics, or to provide marketing information or brand designation. As an alternative to providing specifically demarkated slots or needle receptacles, a large pore foam material can be employed as the body material, provided that suture needles can penetrate the material from a body edge with the use of only light to moderate manual force.

A variety of suturing devices are available for use with disposable suture needles in surgery. In particular, suturing devices for endoscopy procedures utilize pre-threaded needles, and there is a frequent need to replace the needle during surgery, for example once the suture attached to the needle has been used up. Typically, the device is withdrawn from the region of suturing, and the used needle must be removed, stored for counting, discarded as hazardous waste, and replaced. A number of such suturing devices are described in U.S. Pat. No. 5,645,552, which is hereby incorporated by reference. A commonly used device is the Endo Stitch™ device sold by the Autosuture division of United States Surgical Corporation. Of particular importance is the ability to grasp the needle without the need to manually grasp the needle. Thus, the user's hand and fingers are not exposed to the risk of needle puncture during removal after they have been contaminated during use.

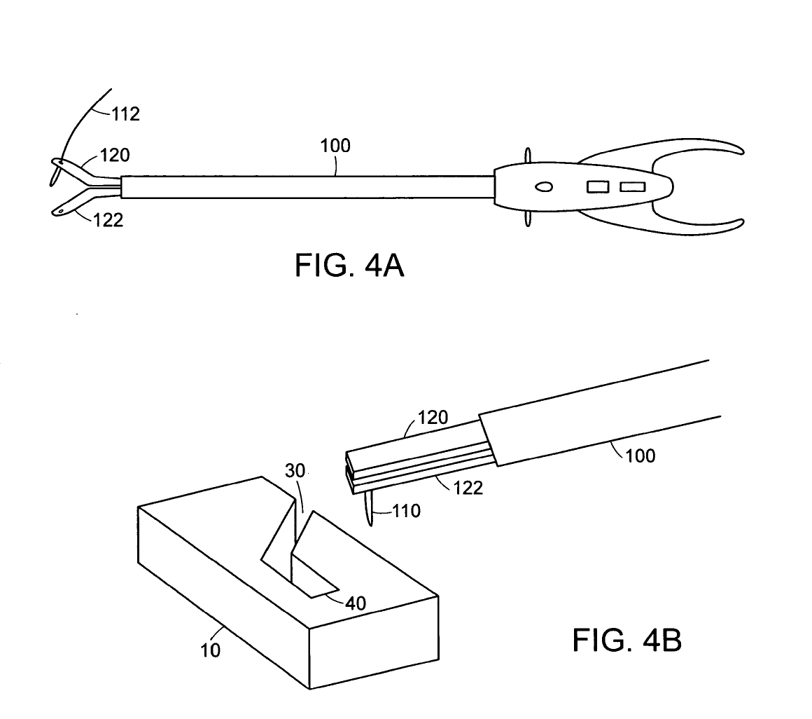

One embodiment of a suturing device for use with the present invention is depicted in FIG. 4A. The suturing device 100 has two arms or jaws, each of which terminates in an attachment site for either an upper holder 120 or a lower holder 122 for a suture needle 110 which is attached to a suture 112. The jaws of the device alternately grasp the needle, and the user can actuate a mechanism to transfer the needle grasping mechanism from one arm to the other as a region of a tissue or organ is sutured. In FIG. 4B, the arms of the suturing device 100 are depicted in juxtaposition with a slot 30 in the body 10 of a needle storage device. The needle 110 is passed into slot 30 by movement of the body and the suturing device relative to each other to bring the needle storage device between the arms of the suturing device which are typically separated by less than 30 mm. Once the needle enters the slot, the needle is slid down into the slot, any bends or angles are then negotiated, and the needle is parked in the needle receptacle 40. At this time, an actuator of the suturing device can be triggered to release the needle. In a preferred embodiment, the jaws of the suturing device are in a closed configuration while the needle is positioned in the storage device; once the needle is positioned for storage the jaws of the suturing device are opened and the needle is released. The storage device is then withdrawn from the arms of the suturing device. The body can include a housing, a foam inner body and a moveable cover as described previously herein.

While the invention has been described in connection with specific methods and apparatus, those skilled in the art will recognize other equivalents to the specific embodiments herein. It is to be understood that the description is by way of example and not as a limitation to the scope of the invention and these equivalents are intended to be encompassed by the claims set forth below.

What is claimed is:

1. A low profile storage device for suture needles, the device comprising a body having a body portion with a thickness and having one or more slots opening at an edge of said body, each of said slots having a size to hold a suture needle.

2. The storage device of claim 1, wherein at least one slot comprises two or more joined segments, and each pair of joined segments is connected at an angle.

3. The storage device of claim 2, wherein at least one slot comprises two segments or three segments.

4. The storage device of claim 2, wherein the body portion has a thickness profile fitting between arms of a suturing device.

5. The storage device of claim 2, wherein at least one slot is T-shaped.

6. The storage device of claim 1, wherein at least one slot comprises a central channel connecting with a plurality of side channels, each of said side channels having a size to hold a suture needle.

7. The storage device of claim 1, wherein at least one slot comprises one or more storage positions disposed along its length, each of said storage position having a size to hold a suture needle.

8. The storage device of claim 1, wherein said body is wheel shaped, and a plurality of said slots are radially disposed around a central axis of said body.

9. The storage device of claim 1, wherein said body is fabricated from a foam, plastic, or rubber material.

10. The storage device of claim 9, wherein said material is elastic.

11. The storage device of claim 9, wherein said body is fabricated from a composite of two or more materials.

12. The storage device of claim 11, wherein said body comprises a foam core.

13. The storage device of claim 1, further comprising a closer device.

14. The storage device of claim 13, wherein said closure device is a movable cover.

15. The storage device of claim 13, wherein said cover comprises an opening to the introduce suture needles.

16. The storage device of claim 15, wherein said body is circumferentially shaped, and said cover rotates about the central axis of said body to provide access to a plurality of radially disposed slots.

17. The storage device of claim 1, wherein said suture needles are surgical suture needles having a diameter in a range of 0.1 mm to 2 mm.

18. The storage device of claim 17, wherein said suture needles have been used with an endoscopic suturing device.

19. The storage device of claim 1, further comprising a base attached to and supporting said body portion.

20. The storage device of claim 1, wherein a portion of the slot is narrowed to a size less than the needle diameter.

21. The storage device of claim 1, wherein a portion of the slot is coated with an adhesive.

22. The storage device of claim 1 wherein the device has a needle storage position having a width in a range of 0.1 mm to 3.0 mm.

23. The storage device of claim 1 wherein said body has a thickness of less than 30 mm.

24. The storage device of claim 1 wherein the device further comprises a kit.

25. The storage device of claim 24 wherein the kit further comprises a suturing device.

26. The storage device of claim 13 wherein the closer device is movable between an open position and a closed position.

27. The storage device of claim 1 wherein the device can grasp a needle in a slot without manually contacting the needle.

28. The storage device of claim 1 wherein the body comprises a plurality of equally spaced slots.

29. The storage device of claim 1 wherein said body portion has a uniform thickness along a selected cross-section or volume.

30. A method of removing a suture needle from a suturing device comprising:
positioning a storage device adjacent the suturing device;
sliding the suture needle into a slot on the storage device; and
releasing the suture needle from the suturing device.

31. The method of claim 30 further comprising suturing a region with a suturing needle mounted on a suturing device.

32. The method of claim 30 further comprising inserting the storage device between suture needle holder elements of the suturing device.

33. The method of claim 30 further comprising discarding the storage device.

34. The method of claim 30 further comprising opening a cover on the storage device.

35. The method of claim 30 further comprising closing a cover on the storage device.

36. The method of claim 30 wherein at least one slot on the storage device comprises a plurality of side channels or spaces for receiving suture needles.

37. The method of claim 30 further comprising providing the storage device with a plurality of slots.

38. The method of claim 37 further comprising sliding a plurality of suture needles into storage positions of the device.

39. The method of claim 30, wherein a cover of the storage device is repositioned after inserting a suture needle into the storage device.

40. The method of claim 30, further comprising providing the device with a base and said base rests on a support surface.

41. The method of claim 30, wherein an actuator on the suturing device is used to release the suture needle.

42. The method of claim 30, further comprising holding the suture needle in the storage device by pressure of body material against the needle.

43. The method of claim 42, further comprising compressing the body material with the needle.

44. The method of claim 30, further comprising holding the suture needle in the storage device by an adhesive coating on said body.

45. The method of claim 32 further comprising providing a suturing device having a pair suture needle holder elements separated by less than 30 mm.

46. The method of claim 30 further comprising providing a storage device having a housing, a foam inner body and a cover.

47. The method of claim 30 further comprising grasping a needle with the storage device without manually contacting the needle.

48. The method of claim 30 further comprising inserting a body portion having a uniform thickness between arms of a suturing device to remove a needle mounted on the suturing device.
